(19) United States
(12) Patent Application Publication
(10) Pub. No.: US 2002/0110902 A1
(43) Pub. Date: Aug. 15, 2002

(54) Robotic autosampler for automated electrospray from a microfluidic chip

(76) Inventors: Simon J. Prosser, Ithaca, NY (US); Geoffrey S. Rule, Aurora, NY (US); Gary A. Schultz, Ithaca, NY (US); Thomas N. Corso, Lansing, NY (US)

Correspondence Address: Michael L. Goldman, NIXON PEABODY LLP, Clinton Square, P.O. Box 31051, Rochester, NY, 14603-1051, US

(21) Appl. No.: 10/058,533

(22) Filed: Jan. 28, 2002

Related U.S. Application Data
(60) Provisional application No. 60/264,501, filed on Jan. 26, 2001.

(51) Int. Cl. C12M 1/34

(52) U.S. Cl. 435/287.1

(57) ABSTRACT

A robotic autosampler provides for automated manipulation of microfluidic chips having multiple electrospray devices and/or sample inlets for interface to a mass spectrometer or other detection device. The autosampler also provides for connection of control voltages to the electrospray device to facilitate enablement, control and steering of charged droplets and ions. The autosampler further provides a method of fluid delivery that may be disposable or reusable. The delivery device may contain materials for component separation or sample purification. The delivery device may contain preloaded sample or the sample may be loaded by the autosampler. A method for automated manipulation of multiple electrosprays in communication with a detector, includes: providing a robot autosampler having an electrospray chip; electrospraying at least one analyte from at least one electrospray device on the electrospray chip; and manipulating the electrospray chip in communication with a detector in a manner to detect analyte from the electrospray.

### FIELD OF THE INVENTION

The present invention relates to a robotic autosampler. The robotic autosampler provides for automated manipulation of microfluidic chips having multiple electrospray devices and/or sample inlets for interface to a detection device, such as a mass spectrometer. Multiple samples are brought to the electrospray device to be electrosprayed without any part of the delivery system coming into contact with more than one sample at a time, thus eliminating cross contamination. The apparatus also provides for connection of control voltages to the electrospray device to facilitate enablement, control and steering of charged droplets and ions.

### BACKGROUND OF THE INVENTION

Current trends in protein identification, drug discovery, and drug development, are creating new demands on analytical techniques. For example, the use of mass spectrometry to identify known, and sequence unknown proteins is undergoing very rapid growth in efforts to identify new drug targets and identify markers of disease states. The effort to characterize all of the proteins in whole organisms (proteomics) is a natural progression from the genome sequencing efforts of the past decade but may be an even greater undertaking. One reason for this is the large number of different post-translational modifications proteins may undergo. Modifications such as phosphorylation, glycosylation, acetylation and ubiquitination may occur at several sites on a protein, tremendously increasing the number of possible forms and oftentimes altering the biological function of the protein. Consequently, in addition to routine identification of proteins after enzymatic digestion, a large part of current proteomics effort is directed towards determining the sites and types of amino acid modifications on proteins of interest.

Nanoelectrospray mass spectrometry is the method of choice for determination and characterization of low abundance proteins. This technique, developed by Wilm and Mann Int. J. Mass Spectrom, Ion Processes 136:167-180 (1994) and Anal. Chem. 68:1-8 (1996), provides high sensitivity analyses combined with low sample consumption to provide for long data acquisition times and multiple experiments on precious samples. For example, at a 100 nL/min flow rate a 5 μL sample can be expected to last for 50 minutes. This allows the analyst to perform multiple experiments on the mass spectrometer followed by database searches for possible protein identification or, failing identification, additional experiments for de novo sequencing of the protein. Up to this time the process of performing nanoelectrospray mass spectrometry has involved manual manipulation of individual pulled capillary tips. These tips are time consuming to prepare and difficulties arise when samples require transfer to a new tip due to tip blockage.

Current trends in drug discovery and development are also creating new demands on analytical techniques. For example, combinatorial chemistry is often employed to discover new lead compounds, or to create variations of a lead compound. Combinatorial chemistry techniques can generate thousands of compounds (combinatorial libraries) in a relatively short time (on the order of days to weeks). Testing such a large number of compounds for biological activity in a timely and efficient manner requires high-throughput screening methods which allow rapid evaluation of the characteristics of each candidate compound.

The quality of the combinatorial library and the compounds contained therein is used to assess the validity of the biological screening data. Confirmation that the correct molecular weight is identified for each compound or a statistically relevant number of compounds along with a measure of compound purity are two important measures of the quality of a combinatorial library. Compounds can be analytically characterized by removing a portion of solution from each well and injecting the contents into a separation device such as liquid chromatography or capillary electrophoresis instrument coupled to a mass spectrometer.

Development of viable screening methods for these new targets will often depend on the availability of rapid separation and analysis techniques for analyzing the results of assays. For example, an assay for potential toxic metabolites of a candidate drug would need to identify both the candidate drug and the metabolites of that candidate. An understanding of how a new compound is absorbed in the body and how it is metabolized can enable prediction of the likelihood for an increased therapeutic effect or lack thereof.

Given the enormous number of new compounds that are being generated daily, improved systems for identifying molecules of potential therapeutic value for drug discovery are being developed. Microchip-based separation devices have been developed for rapid analysis of large numbers of samples. Compared to other conventional separation devices, these microchip-based separation devices have higher sample throughput, reduced sample and reagent consumption, and reduced chemical waste. The liquid flow rates for microchip-based separation devices range from approximately 1-500 nanoliters per minute for most applications. Examples of microchip-based separation devices include those for capillary electrophoresis (“CE”), capillary electrochromatography (“CEC”) and high-performance liquid chromatography (“HPLC”) include Harrison et al., Science 261:859-97 (1993); Jacobson et al., Anal. Chem. 66:1114-18 (1994), Jacobson et al., Anal. Chem. 66:2369-73 (1994), Kutter et al., Anal. Chem. 69:5165-71 (1997) and He et al., Anal. Chem. 70:3790-97 (1998). Such separation devices are capable of fast analyses and provide improved precision and reliability compared to other conventional analytical instruments.

Still faster and more sensitive systems are being designed to provide high-throughput screening and identification of compound-target reactions in order to identify potential drug candidates. Examples of such improved systems include those disclosed in U.S. patent application Ser. No. 09/748,518, entitled “Multiple Electrospray Device, Systems and Methods,” filed Dec. 22, 2000 and U.S. patent application Ser. No. 09/764,698, entitled “Separation Media, Multiple Electrospray Nozzle System and Method,” filed Jan. 18, 2001, which are each incorporated herein in their entirety.

The potential array size, high-throughput, and speed improvements over conventional technology that such devices offer can be facilitated with suitable automation of these devices. Thus, there is a need for automated manipulation of microfluidic chips having multiple electrospray devices and/or sample separation inlets for interface to a detection device, such as a mass spectrometer.

### SUMMARY OF THE INVENTION

The present invention relates to a robot autosampler including:

a probe carriage being movable between a sample source and an electrospray chip holder and including a fluid delivery probe which accepts sample from the source and discharges sample to the chip holder;

an electrospray chip holder; and

an alignment mechanism which aligns the probe with the chip holder and the chip holder with a detector.

Another aspect of the present invention allows the fluid delivery probe to rotate through 90 degrees so that it may address multiple samples, for example in 96- or 384-well sample plates, and arrays of sample loading devices such as pipette tips, syringe tips or capillary tubes. An internal syringe pump adds the ability to aspirate samples into the tips/tubes by creating a partial vacuum. In this way the invention may serially pick up samples in disposable tips that are sealed against the back of the electrospray device thus fully automating not only the electrospray technique but also sample handling. Use of a fresh tip/tube and electrospray nozzle for each sample ensures that there is no cross contamination between samples.

Another aspect of the present invention relates to a voltage probe electrically insulated from and mounted to the fluid delivery probe.

A further aspect of the present invention relates to an electrospray chip mounted to the chip holder.

Another aspect of the present invention relates to a detector in electrospray communication with the electrospray chip. The detector can be a mass spectrometry device.

Another aspect of the present invention relates to a method for automated manipulation of multiple electrosprays in communication with a detector including providing the robot autosampler noted above, electrospraying at least one analyte from at least one electrospray device on the electrospray chip and manipulating the electrospray chip in communication with a detector in a manner to detect analyte from the electrospray.

Another aspect of the present invention relates to a method for automated manipulation of multiple samples for generation of multiple electrosprays in communication with a detector, including:

providing a robot autosampler, which can be programmed to engage a tip onto a fluid delivery probe, load the tip with sample containing at least one analyte, transfer the sample loaded tip to communicate with an electrospray chip containing at least one electrospray device, electrospray the at least one analyte, discard the used tip, and engage another tip onto the probe to repeat the loading, transferring, and electrospraying cycle;

engaging a tip onto the autosampler probe;

loading the probe tip with a sample containing at least one analyte;

transferring the at least one analyte to at least one electrospray device on the electrospray chip;

electrospraying the at least one analyte from at least one electrospray device on the electrospray chip;

manipulating the electrospray chip in communication with a detector in a manner to detect analyte from the electrospray, and repeating the engaging, loading, transferring, and electrospraying cycle.

### BRIEF DESCRIPTION OF THE DRAWINGS

FIG. 1 is a perspective view from one side of a robotic autosampler in accordance with one embodiment of the present invention with a probe carriage assembly in position to address a chip;

FIG. 2 is a partial, perspective view from the one side of the robotic autosampler with the probe carriage assembly in a rotating position;

FIG. 3 is a perspective view from the one side of the robotic autosampler with the probe carriage assembly in position to address a sample;

FIG. 4 is a perspective view from another side of the robotic autosampler to show the probe carriage cam track;

FIG. 5 is a perspective view from the other side with a portion of the robotic autosampler removed to show the probe carriage cam track;

FIG. 6 is a cross-sectional view of the probe carriage assembly;

FIG. 7 is a perspective view of the probe carriage assembly engaging a tip ejection assembly;

FIG. 8 is a partial, perspective view from yet another side of the robotic autosampler to show the chip holder assembly;

FIG. 9 is a partial, perspective view of a cutaway portion of another embodiment of the robotic autosampler to show the chip holder assembly and a platform adjustment assembly;

FIG. 10 is a perspective view of the relative movement capabilities of certain components of the robotic autosampler;

FIG. 11 is a cross-section view of application of voltage to the fluid by the fluid probe;

FIG. 12 is a cross-section view of application of voltage to the fluid by use of a voltage probe in contact with a conducting surface of the electrospray ionization (“ESI”) chip;

FIG. 13 is a top plan view of the chip circuitry in which voltage is applied individually to any number of electrospray devices at the same time, individually, or in groups;

FIG. 14 is a cross-section view of an electrospray ionization chip having electrodes in which voltage is applied to all electrospray devices on the chip at the same time;

FIG. 15 is a cross-section view of an electrospray ionization chip holder providing voltage to the chip;

FIG. 16A is a cross-section view of an electrospray ionization chip having annulus electrodes;

FIG. 16B is a cross-section view of an electrospray ionization chip having surface electrodes; and

FIG. 16C is a cross-section view of an electrospray ionization chip having stacked electrodes.

### DETAILED DESCRIPTION OF THE INVENTION

The present invention relates to a robot autosampler, having a fluid delivery probe carriage which engages a pipette tip, loads sample into the pipette tip, and places the sample-loaded pipette tip probe in communication with an electrospray chip. Optionally, the pipette tip is pre-loaded with sample. The electrospray chip is placed in communication with a detection device which analyses the sprayed analyte sample. The probe carriage includes a syringe pump connected to the probe by an air-tight connection. The probe carriage removes sample from the sample tray, loads the pipette tip with sample and expels sample from the pipette tip to the chip. In one embodiment, the autosampler provides electrical current to the chip. The autosampler electrosprays the sample into a detection device, for example, a mass spectrometer. After spraying, the used pipette tip is discarded and a new pipette tip is picked up to start another cycle. The autosampler includes a pipette tip tray which holds a plurality of pipette tips and a sample tray which contains a plurality of samples. In another embodiment, the autosampler includes a pipette tip tray wherein the pipette tips are pre-loaded with sample. A chip holder is mounted on the autosampler which places the chip in communication with the detection device.

The present invention also relates to a method for automated manipulation of multiple electrosprays in communication with a detector, including: providing a robot autosampler which can engage a probe tip, load the tip with sample, transfer the sample to an electrospray chip; electrospraying at least one analyte from at least one electrospray device on the electrospray chip; and manipulating the electrospray chip in communication with a detector in a manner to detect analyte from the electrospray. Optionally, the engaged probe tip has been pre-loaded with sample.

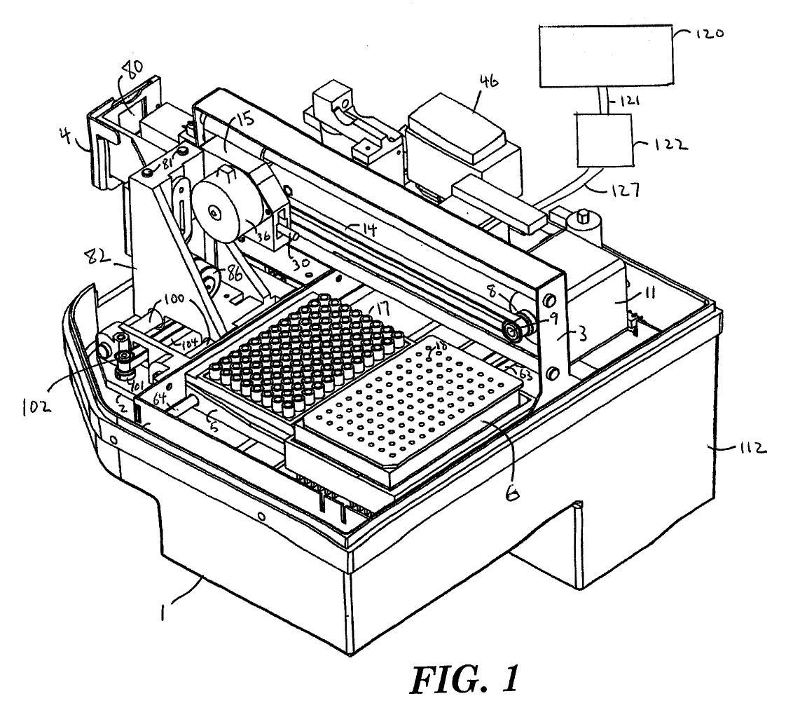

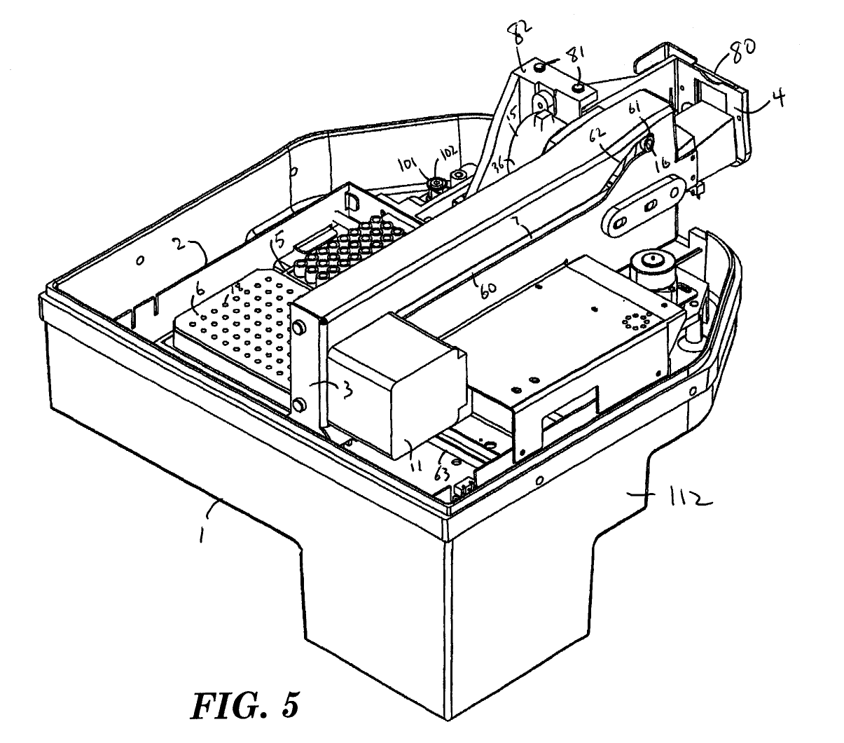

Referring to FIGS. 1-5, the autosampler 1 includes a housing 2 with a bracket 3 which extends along a Z-axis adjacent a chip holder 4, a pipette tray 5 including tips 17 and a sample tray 6 including sample wells 18 in this particular example. A track 7 with three sections extends along a top portion of the bracket 3, although the number of sections of track 7 can vary. An idler roller 12 is rotatably mounted on a shaft 10 extending from the bracket 3. A rotatable drive shaft 9 is connected to a probe carriage motor 11. A drive roller 8 is mounted to the drive shaft 9. A belt 14 is seated over the idler roller 12 and drive roller 8 and extends along the Z-axis. The probe carriage motor 11 is connected to rotate the drive shaft 9 in two directions depending on the desired movement of a probe carriage 15.

The probe carriage 15 includes a probe carriage drive system (not shown) with a cam follower 16, although the probe carriage drive system can include other and/or different components. The cam follower 16 extends from the probe carriage 15 and is seated in the track 7 for movement along the track 7. The probe carriage drive system is connected to the belt 14, for example by a belt clamp, to move the probe carriage 15 along the Z-axis.

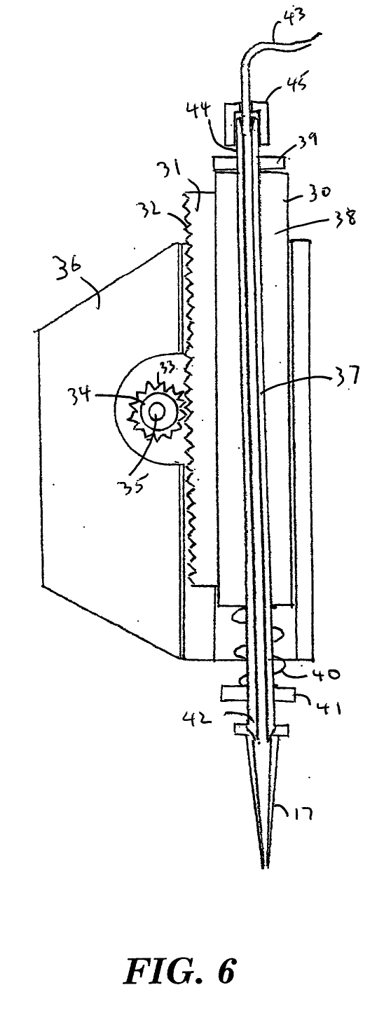

The probe carriage 15 also includes a probe 30 connected to a probe rack 31, as shown in FIG. 6. Although one probe is shown in this embodiment, a plurality of probes can be mounted on the probe carriage in a similar manner. The probe rack 31 includes teeth 32 meshed with teeth 33 of a probe drive gear 34. The probe drive gear 34 is mounted to a rotatable drive shaft 35 connected to a probe motor 36. The probe motor 36 is connected to rotate the drive shaft 35 in two directions depending on the desired movement of the probe 30. The probe 30 includes a hollow tube 37 slideably held within a cylindrical probe insulator 38 at one end by a first retaining collar 39 and at the other end by a spring 40 circumscribing the tube 37 and extending between the probe insulator 38 and a second retaining collar 41 positioned to tension the hollow tube 37 in opposing directions. A tip 17 is attached to the spring-loaded end of the probe 30, which can be a pipette tip or other tip. The probe end 42 is shaped to insert into and attach to one end of the tip 17. A flexible tube 43 is attached to the other end 44 of the hollow tube 37 by a compression fitting 44 to form an air-tight seal. The other end of the flexible tubing is attached to a syringe pump (not shown) to provide a partial vacuum within the tube and to an adjustable pressure regulator 46 to provide positive pressure to expel the sample. The syringe pump and pressure regulator 46 are connected to the flexible tubing by two valves which can be activated to switch between each.

The syringe pump may include any number of commercially available syringe pumps. Conventional syringe pumps known in the art suitable for practice of the present invention include pipetters which generate a partial vacuum by displacing a plunger to increase volume and thus reduce pressure so the liquid is drawn into the tip and those described in “Small Volume Pipetting”, T. W. Astie Journal of the Association of Laboratory Automation (JALA), Vol. 3, No.3, 1998, which is incorporated herein in its entirety.

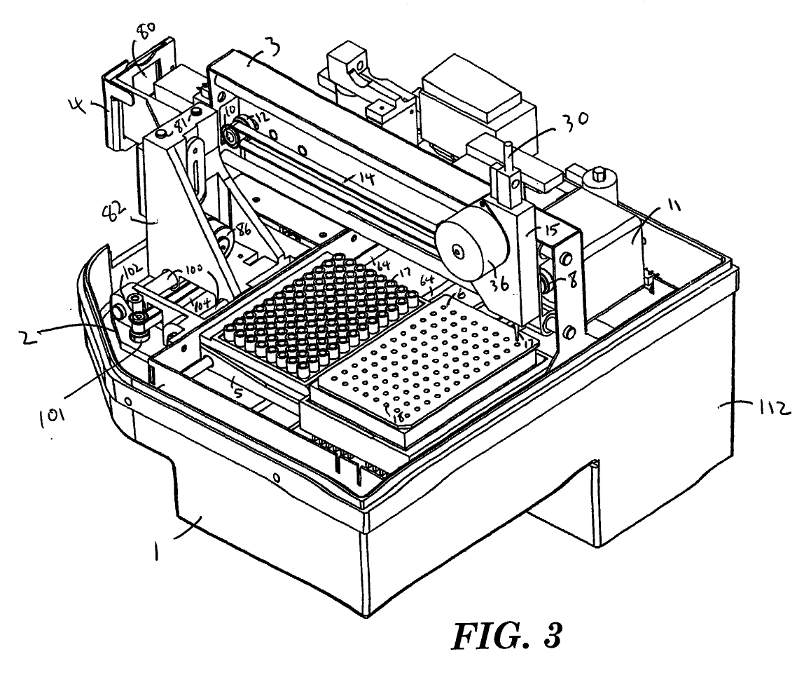

A first section 60 of the track 7, as shown in FIGS. 3-5, is adjacent the pipette tray 5 and sample tray 6, in this example. Optionally, the pipette tray 5 can include pipettes 17 pre-loaded with sample 110 and the first section 60 is adjacent the pipette tray 5 containing the pre-loaded tips. The syringe pump or other liquid pump can provide fluid to deliver sample to the chip. The first section 60 of the track 7 forms a line parallel with the Z-axis. A third section 61 of the tract 7, as shown in FIGS. 1, 4 and 5, is adjacent the chip holder 4 and forms a line parallel with the Z-axis. A second section 62 of the tract 7 is interposed between the first section 60 and third section 61. The second section 62 circumscribes a 90° arc in the Z-Y plane. The cam follower 16 is connected to the probe carriage 15 to maintain the probe 30 parallel with the Y-axis when the probe carriage 15 moves along the first section 60 of the track 7 and to maintain the probe 30 parallel with the Z-axis when the probe carriage 15 moves along the third section 61 of the track 7. When the probe carriage 15 moves along the second section 62 of the track 7, the cam follower 16 circumscribes a 90° arc in the Z-Y plane transitioning the probe 30 between a position parallel with the Z-axis and a position parallel with the Y-axis.

The sample tray 6 is slideably mounted in the autosampler housing 2 on a pair of support shafts 63. The sample tray 6 includes a plurality of sample wells 18, for example, standard 96-well sample or 384-well sample plates. An idler roller (not shown) is rotatably mounted on a shaft (not shown) extending from the housing 2. A rotatable drive shaft (not shown) is connected to a sample tray motor (not shown). A drive roller (not shown) is mounted to the drive shaft. A belt (not shown) is seated over the idler roller and drive roller and extends along the X-axis. The sample tray motor is connected to rotate the drive shaft in two directions depending on the desired movement of the sample tray 6. The sample tray 6 includes a sample tray drive system (not shown), although can include other and/or different components. The sample tray drive system is connected to the belt, for example by a belt clamp, to move the sample tray along the X-axis.

The pipette tip tray 5 is slideably mounted in the autosampler housing 2 on a pair of support shafts 64. The pipette tip tray 5 includes a plurality of pipette tips 17, for example, a standard 96 pipette tip tray. An idler roller (not shown) is rotatably mounted on a shaft (not shown) extending from the housing 2. A rotatable drive shaft (not shown) is connected to a pipette tip tray motor (not shown). A drive roller (not shown) is mounted to the drive shaft. A belt (not shown) is seated over the idler roller and drive roller and extends along the X-axis. The pipette tip tray motor is connected to rotate the drive shaft in two directions depending on the desired movement of the pipette tip tray 5. The pipette tip tray 5 includes a pipette tip tray drive system, although can include other and/or different components. The pipette tip drive system is connected to the belt, for example by a belt clamp, to move the sample tray along the X-axis.

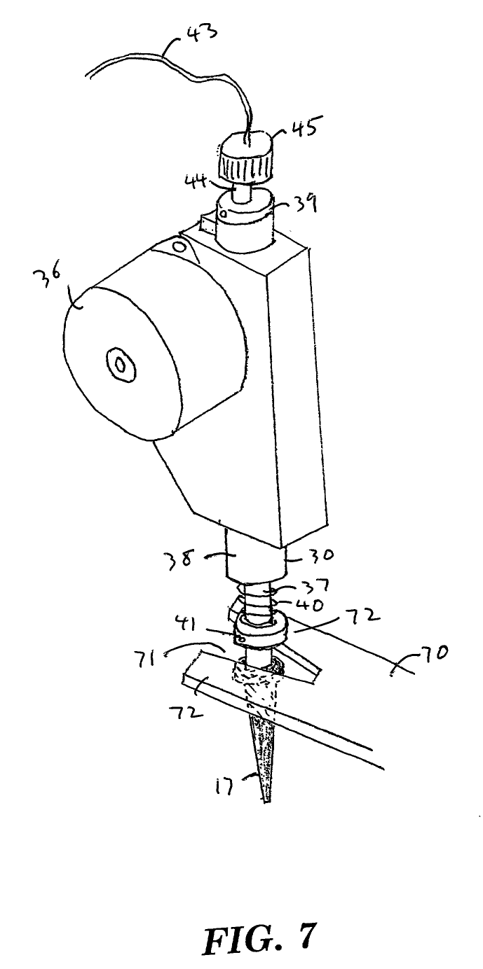

As shown in FIG. 7, an ejector plate 70 is connected to the sample tray 6 adjacent to the track 7. The ejector plate 70 has a v-shaped forked notch 71 positioned to engage with the pipette tip 17 of the probe 30 when activated. The tines 72 of the notch 71 are positioned along the Z-axis and transverse to the direction of travel of the probe 30 when the probe motor 36 is activated.

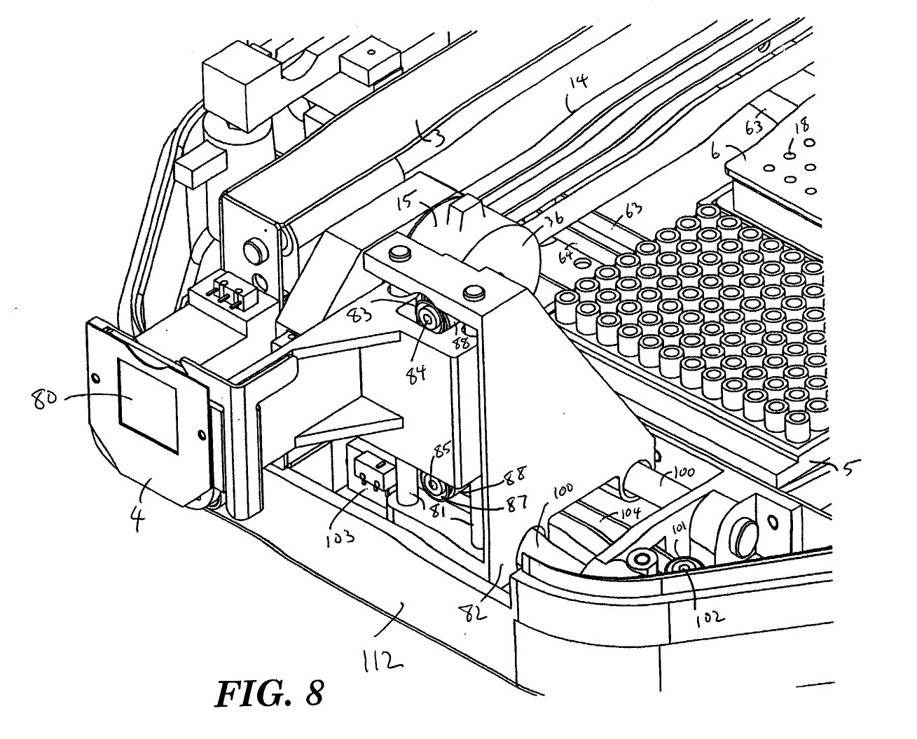

As shown in FIG. 8, an electrospray chip 80 is mounted to the chip holder 4. The chip holder 4 is slideably mounted on a pair of support shafts 81 to a chip holder housing 82. An idler roller 83 is rotatably mounted on a shaft 84 extending from the chip holder housing 82. A rotatable drive shaft 85 is connected to a chip holder motor 86. A drive roller 87 is mounted to the drive shaft 85. A belt 88 is seated over the idler roller 83 and drive roller 87 and extends along the Y-axis. The chip holder motor 86 is connected to rotate the drive shaft 85 in two directions depending on the desired movement of the chip holder 4. The chip holder 4 includes a chip holder drive system (not shown), although can include other and/or different components. The chip holder drive system is connected to the belt 88, for example by a belt clamp, to move the chip holder along the Y-axis.

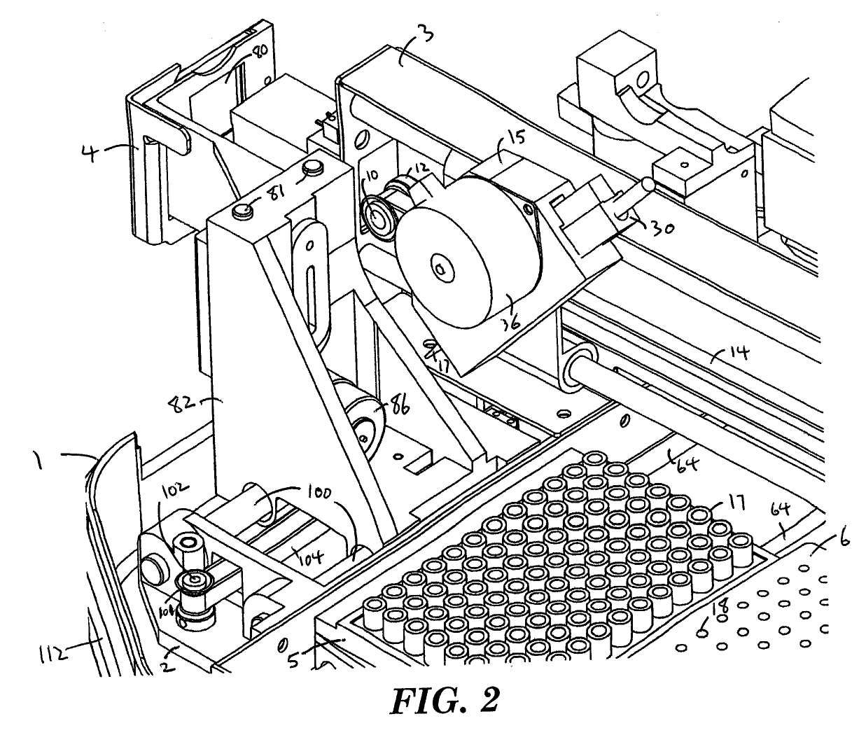

As shown in FIGS. 2 and 8, the chip holder housing 82 is slideably mounted on a pair of support shafts 100 to the autosampler housing 2. An idler roller 101 is rotatably mounted on a shaft 102 extending from the chip holder housing 82. A rotatable drive shaft (not shown) is connected to a chip holder housing motor 103. A drive roller (not shown) is mounted to the drive shaft. A belt 104 is seated over the idler roller 101 and drive roller and extends along the X-axis. The chip holder housing motor 103 is connected to rotate the drive shaft in two directions depending on the desired movement of the chip holder housing 82. The chip holder housing 82 includes a chip holder housing drive system (not shown), although can include other and/or different components. The chip holder housing drive system is connected to the belt 104, for example by a belt clamp, to move the chip holder housing 82 along the X-axis.

Preferably, the chip holder and chip holder housing motors have a resolution of less than ten micrometers. The alignment overall accuracy is preferably greater than 40 micrometers. Pipette tips within this tolerance are typically not commercially available. In such case an alignment mechanism is preferred to correct for tolerance limitations in the pipette tips that would exceed the preferred specifications. A suitable alignment mechanism includes a mechanical device that moves the tip end into correct position. An alignment mechanism (not shown) is mounted to bracket 3 between the chip holder 4 and the probe carriage 15. The alignment mechanism is an aperture in a plate positioned relative to the center of the probe tip when parallel to the Z-axis to correct for any manufacturing variance of the tip.

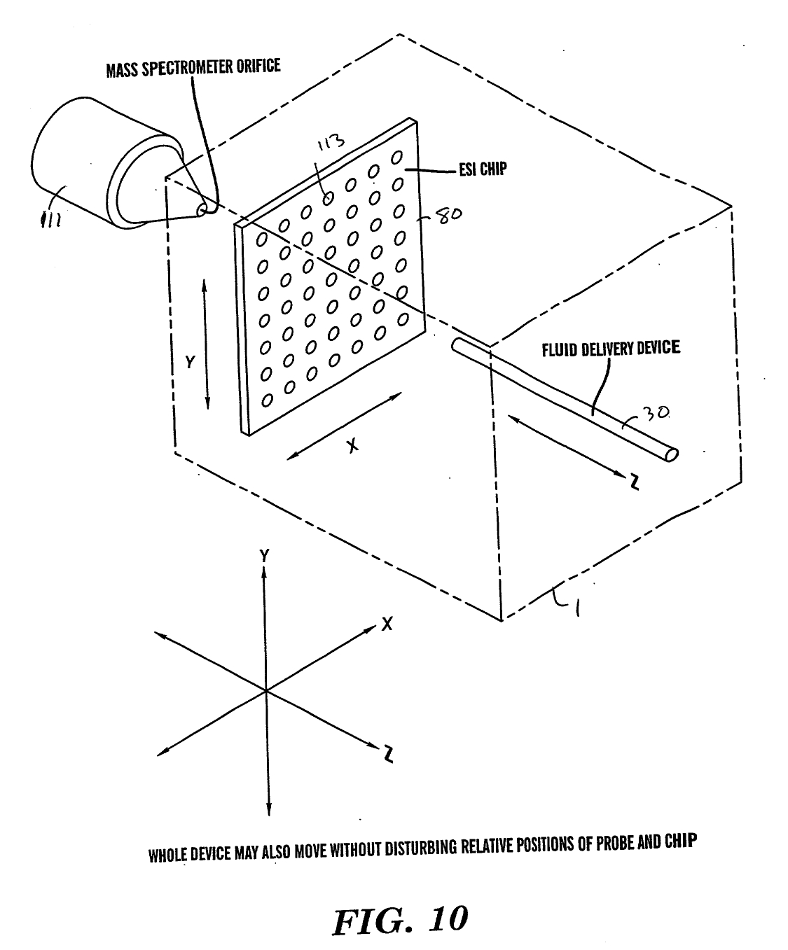

The chip holder 4, chip holder housing 82, probe 30, probe carriage 15, pipette tip tray 5, bracket 3, and sample tray 6 system are mounted within the autosampler housing 2 and connected to a motor (not shown) by a rack and pinion connection (not shown) to move the system along the X-axis depending upon the desired position of the chip 80 with respect to the detector 111 without moving the outside casing 112 of the autosampler device 1. This system is also connected to a motor (not shown) by a rack and pinion connection to move the system along the Y-axis depending upon the desired position of the chip 80 with respect to the detector 111 without moving the outside casing 112 of the autosampler device 1, as shown in FIG. 10.

As shown in FIG. 1, an assembler control system 120 is coupled by electrical leads 121 to a controller box 122. The controller box includes a microprocessor, power supply for the drive motors, control voltages and electrospray voltages for the electrospray chip. The assembler control system 120 controls the drive motors according to the desired sample analysis sequencing. The controller box 122 is coupled to the autosampler 1 by electrical leads 127 which are connected to the drive motors, chip, and probe of the autosampler 1. The assembler control system 120 includes a central processing unit (CPU) or processor, a memory, a graphical user interface or display, and a user input device which are coupled together by a bus system or other link, respectively, although the assembler control system may comprise other components, other numbers of the components, and other combinations of the components.

The processor may execute one or more programs of stored instructions for a method for automated manipulation of multiple samples for generation of multiple electrosprays in communication with a detector in accordance with one embodiment of the present invention as described herein. In this particular embodiment, the programmed instructions executed by CPU are stored in memory, although some or all of those programmed instructions could be stored and retrieved from and also executed at other locations.

A variety of different types of memory storage devices, such as a random access memory (RAM) or a read only memory (ROM) in the system or a floppy disk, hard disk, CD ROM, or other computer readable medium which is read from and/or written to by a magnetic, optical, or other reading and/or writing system that is coupled to the processor, can be used for memory. The graphical user interface provides a display of the information to the operator, such as a sample, pipette tip and chip location data. A variety of different types of displays can be used such, such as a cathode ray tube display device. The user input device enables an operator to generate and transmit signals or commands to the CPU, such as sample selection and chip location. A variety of different types of user input devices can be used, such as a keyboard, keypad, on-screen touch pad, or computer mouse.

In operation, the probe carriage 15 moves along the Z-axis by activation of the probe carriage motor 11 and to start the analysis cycle is initially suspended over a pre-selected one of the pipette tips 17 of the pipette tray 5. The movement of the probe 30 is activated by the probe motor 36 and the probe 30 moves along the Y-axis to extend and engage with the pre-selected pipette tip 17 and attaches the pipette tip 17 to the end 42 of the probe 30. The probe motor 36 is reversed to retract the probe 30 within the probe carriage 15 along the Y-axis and away from the pipette tip tray 5. The probe carriage 15 is moved along the Z-axis by the probe carriage motor 11 and suspended over a pre-selected sample well 18 of the sample tray 6. The probe motor 36 is activated to extend the probe 30 out of the probe carriage 15 along the Y-axis and place the pipette tip 17 in contact with the sample solution 110.

The syringe pump is activated to create a partial vacuum and withdraw sample 110 from the selected sample tray well 18 into the pipette tip 17. The probe 30 is retracted into the probe carriage 15 along the Y-axis by the probe motor 36. The probe carriage 15 is moved along the Z-axis by the probe carriage motor 11 towards the chip holder 4. As the probe carriage 15 nears the chip holder 4, the probe carriage 15 is rotated 90° relative to the Z-axis by the cam follower 16 which reorients the probe 30 from being parallel to the Y-axis to being parallel to the Z-axis.

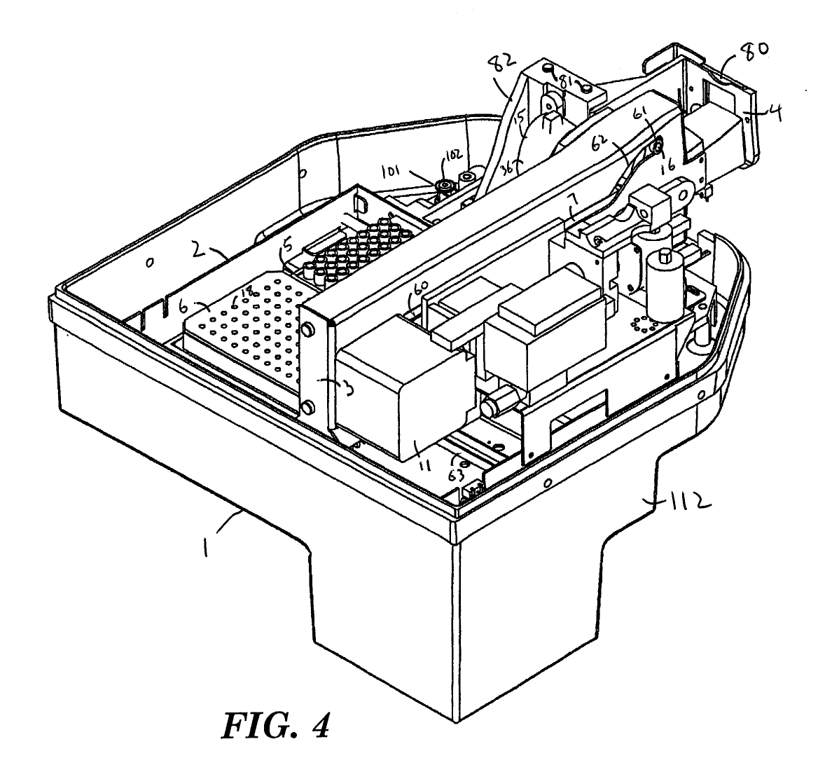

As can be seen in FIGS. 2, 4 and 5, the cam follower 16 is mounted in a track 7 which rotates the probe carriage 15 through 90° relative to the Z-axis at the chip holder 4 end. The probe carriage motor 11 which moves the probe carriage 15 along the Z-axis in the track 7 is shown in FIGS. 3 and 4.

As shown in FIG. 2, when the cam follower 16 of the probe carriage 15 engages the second section 62 of the track 7, the probe carriage 15 rotates through 90° relative to the Z-axis and aligns the probe 30 with the chip holder 4 and parallel to the Z-axis. The probe motor 36 is activated to extend the probe 30 from the probe carriage 15 placing the sample-loaded pipette tip 17 in contact with a pre-selected electrospray receiving well 130 of the chip 80. The pressure regulator is activated to expel sample 110 to the receiving well 130 of the electrospray chip 80 and provide electrical contact to the electrode 114 of the electrospray chip 80 facilitating spraying of the sample 110 into the adjacent detector device 111. After activation, the syringe pump may be used to create a partial vacuum within the pipette tip to draw back any remaining sample to avoid wetting the chip with sample. The probe carriage 15 is moved along the Z-axis by the probe carriage motor 11 in a direction away from the chip holder 4 and rotates 90° along the Z-axis according to the path of the cam follower 16 in the tract 7 to place the probe 30 parallel to the Y-axis.

The pipette tray 5 shown in FIG. 1 is mounted on two parallel shafts 64 and connected to a belt and pulley system driven by a pipette tray motor which moves the pipette tray 5 along the X-axis. An ejector plate 70 is mounted at an edge of the pipette tip tray 5 which is aligned with the probe carriage 15 when the pipette tip tray 5 is moved away from and clears the probe carriage 15 along the X-axis. The probe carriage 15 is moved along the Z-axis by the probe carriage motor 11 and with the probe 30 in the extended position.

As shown in FIG. 7, the pipette tip 17 is removed as the probe carriage 15 moving along the Z-axis engages the ejector plate 70 with the probe 30. The probe 30 is retracted into the probe carriage 15 by the probe motor 36 and the pipette tip 17 engages the fork 71 of the ejector plate 70 and is removed from the probe 30. The probe carriage 15 is now ready to engage a fresh pre-selected pipette tip 17 from the pipette tray S and resume the cycle to analyze the next sample 110. Alternately, the remaining sample in the pipette tip can be returned to the originating sample well to preserve sample, prior to ejecting the tip.

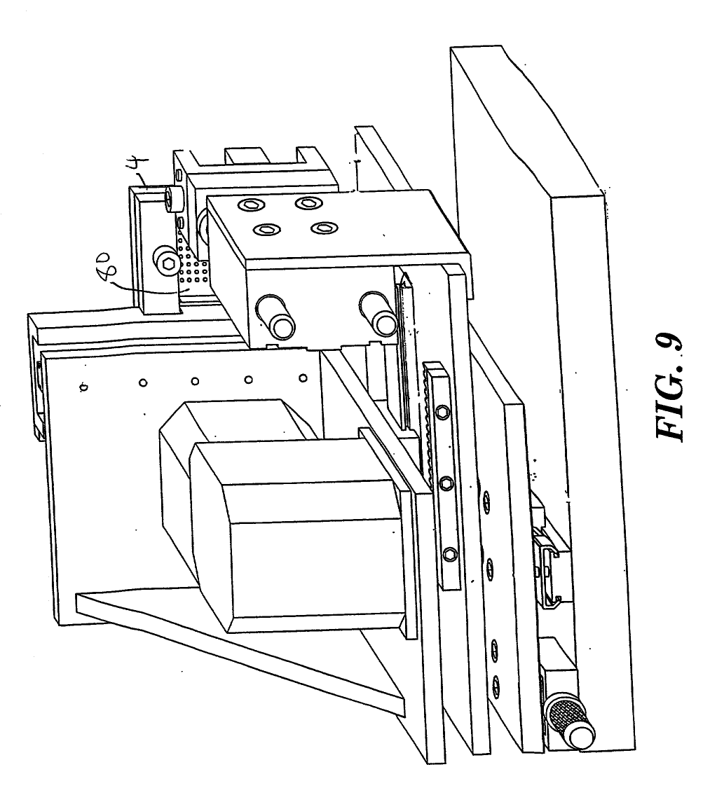

As shown in FIG. 8, the electrospray chip 80 is mounted to a chip holder 4. The chip holder 4 and chip holder housing 82 which can be moved relative to the detector 111 to align the desired electrospray device 115 of the chip 80. The chip holder, chip holder carriage, probe, probe carriage, pipette tip tray, and sample tray are mounted within a housing and connected to motors which can move the system along the X and Y-axis to orient the chip in line with the mass spectrometer 111 without moving the outside casing 112 of the autosampler 1, as shown in FIGS. 9 and 10.

Two stages of motion determine the X and Y-axis position of the chip 80 in front of the mass spectrometer 111 inlet, a third stage of motion moves the probe 30 along the Z-axis over the sample 110 and pipette tip tray 5 and toward the chip 80. As the probe 30 moves along this stage it is held in the Y-Z plane as it traverses the sample 110 and tip tray 5, then the cam follower 16 rotates the probe 90° in the Y-Z plane as it approaches the chip 80. A fourth stage of motion moves the probe along the Y-axis to pick up samples and tips, or along the Z-axis to engage the back of the chip 80 depending on the probe 30 orientation. A fifth stage of motion moves the sample and tip trays 6, 5 under the probe 30 along the X-axis to allow each sample/tip to be indexed by use of this stage in conjunction with the stage which moves the probe 30 along the Z-axis. Two additional stages of motion move the entire assembly along the Z and X-axis to allow optimization of the electrospray position relative to the mass spectrometer inlet. The eighth stage of motion moves a syringe pump to allow samples to be aspirated and dispensed.

All stages of motion are preferably under computer control. This allows for the ability to provide one or a plurality of electrosprays from a grid array of multiple electrospray devices on a microfluidic chip. Preferably, the electrospray chip 80 has a high-density array of electrospray devices 115 or groups of devices 115. Each electrospray device 115 has at least one electrospray outlet 116 and a fluid inlet 113 connected by a channel 117 where the inlet 113 and outlet 116 may either be on the same or opposite sides of the microfluidic chip 80. Preferably, multiple outlets are in fluid communication with a single fluid stream 110.

The X, Y, and Z-axis automated linear motion device is arranged such that a fluid delivery probe can move in the direction of the mass spectrometer orifice. The microfluidic chip is moved relative to the mass spectrometer orifice and fluid delivery probe in the X-axis and Y-axis direction. Thus, the fluid delivery probe remains at a constant X and Y-axis position relative to the mass spectrometer and can move in the Z-axis direction to connect/disconnect the fluid flow that provides the electrospray to the back of the microfluidic chip. The chip remains at a constant Z-axis distance from the orifice of the mass spectrometer and multiple electrospray devices are moved in front of the fluid probe in the X and Y-axis directions so that a grid array of electrospray devices may be electrosprayed sequentially and the electrospray from each may originate from the same point in space.

Other linear motion stages allow for movement of this entire assembly in front of the mass spectrometer. This allows the device to be positioned optimally for maximum performance of the mass spectrometer while the electrospray is active. In the device shown in FIG. 1, there are two stages of movement that provide for movement in the X and Z-axis directions of the fluid probe and chip without moving their positions relative to each other, so that they may be moved while electrospray is occurring for optimization of ion-response of the detector. In conjunction with feedback from the mass spectrometer signal, these stages of movement allow for automation optimization of the position of the electrospray with respect to the detector.

A seal 118 preferably made of a soft material can be used to seal delivery of the fluid 110 to the chip 80. The fluid probe can be sealed against the microfluidic chip using an O-ring or gasket seal. Alternatively, no sealing material is needed when the inlet flow is matched to the demands of the electrospray flow so that fluid is delivered to the inlet at the same rate as the self-sustaining electrospray requirement. Additionally, no sealing material is required when the fluid probe material is capable of forming a direct seal to the chip at the pressure required for efficient electrospray.

The fluid probe may be reusable or disposable so that a new probe is used for each sample and/or electrospray device. The probe may be packed with chromatographic material for component separation or sample purification. The probe may be preloaded with sample or the sample may be delivered in solution to the probe from a reservoir using a suitable pump or other pressure device. The composition of the solution may change over time to help facilitate chromatographic separation. The probe may also deliver a clean solvent to the microfluidic chip, the chip having reservoirs preloaded with sample. The preloaded sample may still be in solution, it may be adsorbed to the chromatographic material of a separation device, or may be in dried form that is resolvated by the solvent delivered by the probe. The chromatographic material/stationary phase may be located in the pipette tip or electrospray chip. Further, multiple fluid probes may be used simultaneously to provide samples to a plurality of electrospray devices.

As the fluid probe moves back to pick up sample, in one embodiment, it moves from the horizontal plane to the vertical plane. The probe may now move up and down to pick up a new pipette tip, or capillary column, or other sample handling device. If sample is not preloaded then the probe can move to a multiple-well sample tray and load sample from a well, before moving back to the chip. Once the sample is sealed against the back of the chip then a small amount of head pressure, typically less than 5 pounds/square inch (“psi”), is provided by the pressure regulator 46 to initiate electrospray. In this way a fresh sample container, and electrospray nozzle may be used for each sample in order to eliminate cross contamination. After analysis the used probe tip/capillary is automatically ejected, for example, by using a mechanical catch, and a fresh probe tip is loaded before aspirating the next sample.

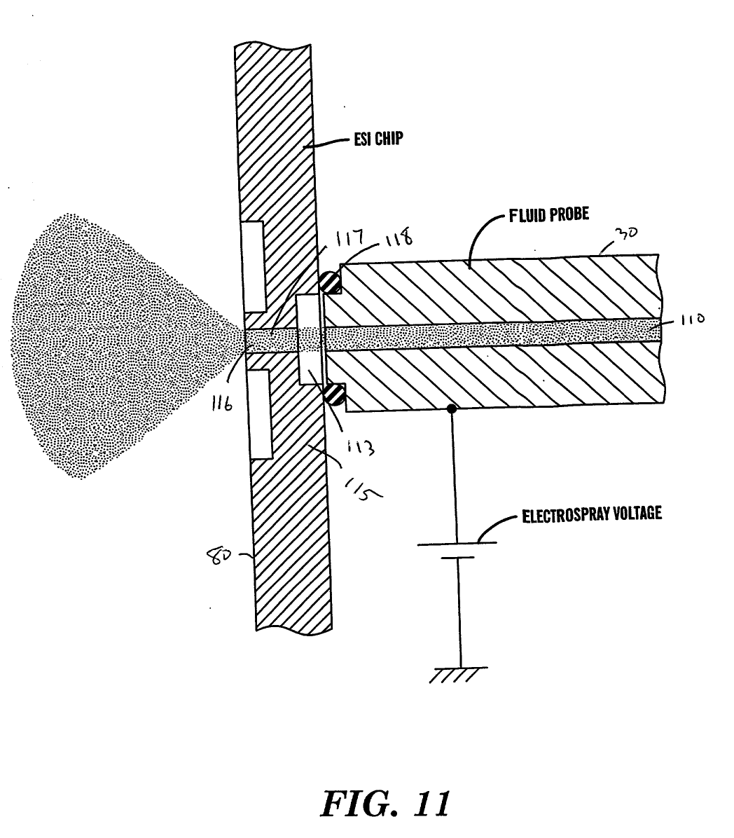

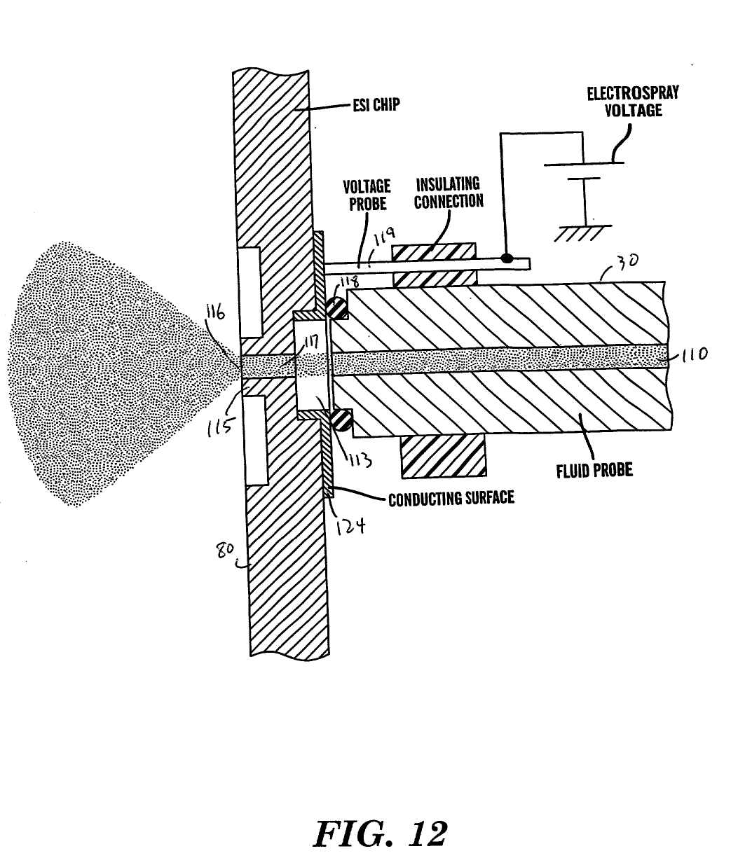

Control voltages for the electrospray are provided either by the microfluidic chip mount or by the fluid delivery probe. The electrospray voltage may be provided by the fluid delivery probe, as shown in FIG. 11, when the probe is electrically conducting, or contacted to the fluid downstream of the probe. Alternatively, this voltage may be provided by an electrically insulated attachment 119 to the probe 30 that makes contact with a conducting surface 123 on the chip 80, as shown in FIG. 12. This has the advantage of providing the voltage at the fluid inlet 113 of the electrospray ionization chip 80 and minimizes electro-osmosis or electrochromatography occurring within the fluid probe 30.

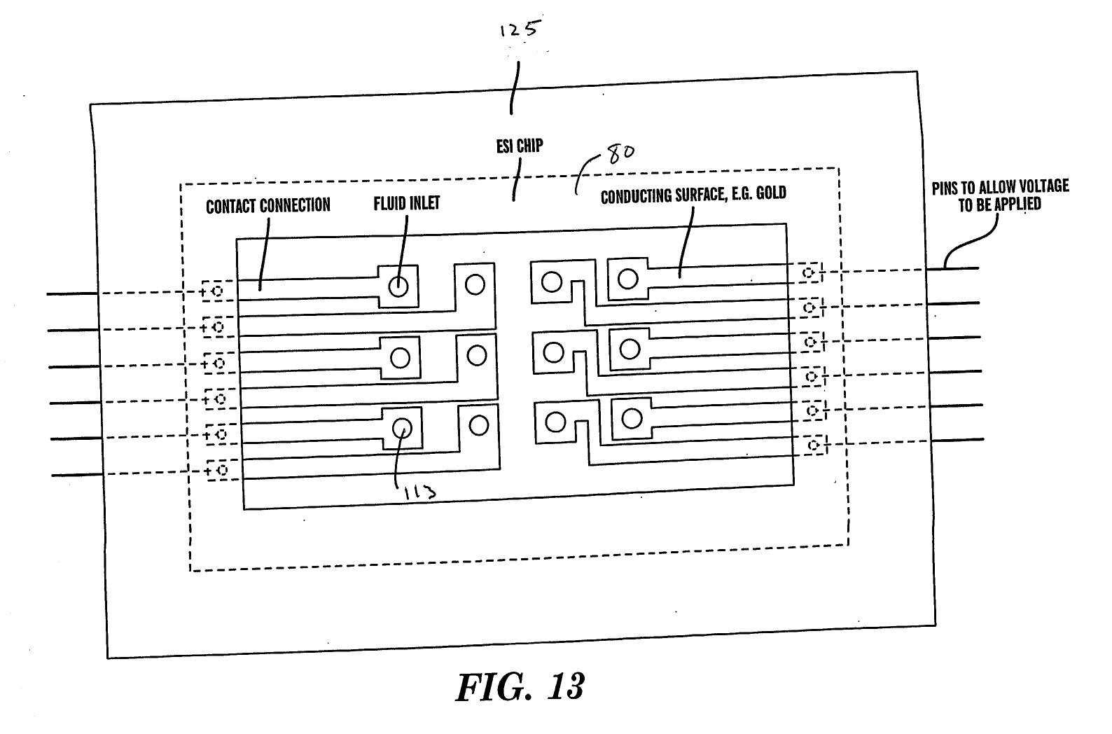

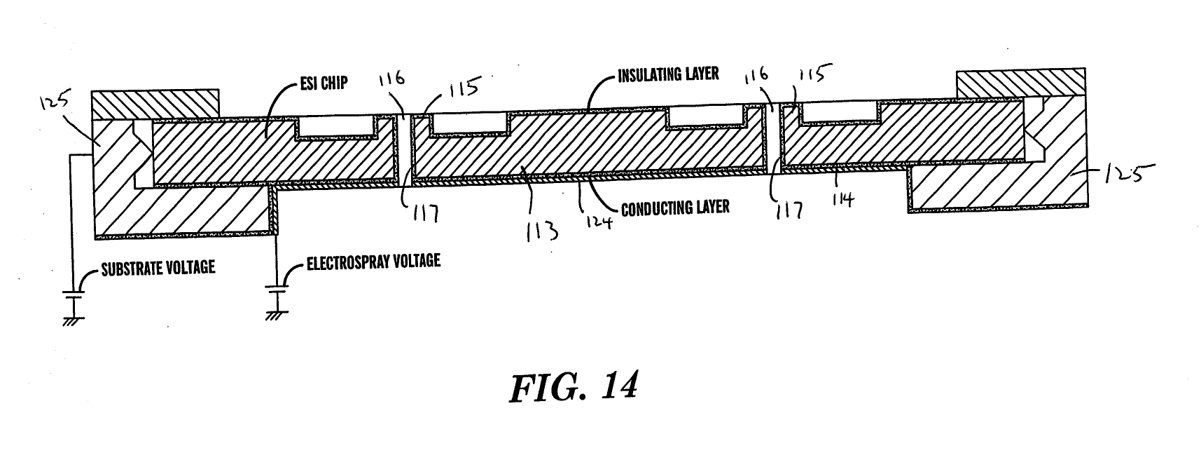

The voltage may also be provided by conducting surfaces 124 extending to the edge of the chip, contacting the chip mount 125 so that voltage may be applied through the chip mount 125. This has the advantage of not needing the probe so that voltage may be applied at any time. Voltage may be applied to any number of electrospray devices at the same time, such as individually, or in groups, as shown in FIG. 13, or all electrospray devices on the chip at the same time, as in FIG. 14, which shows a conducting layer 124 covering the entire inlet surface of the chip.

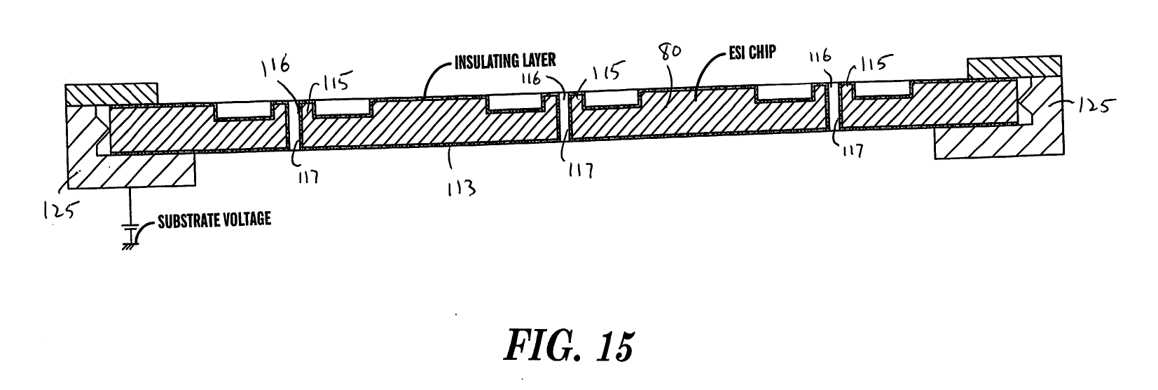

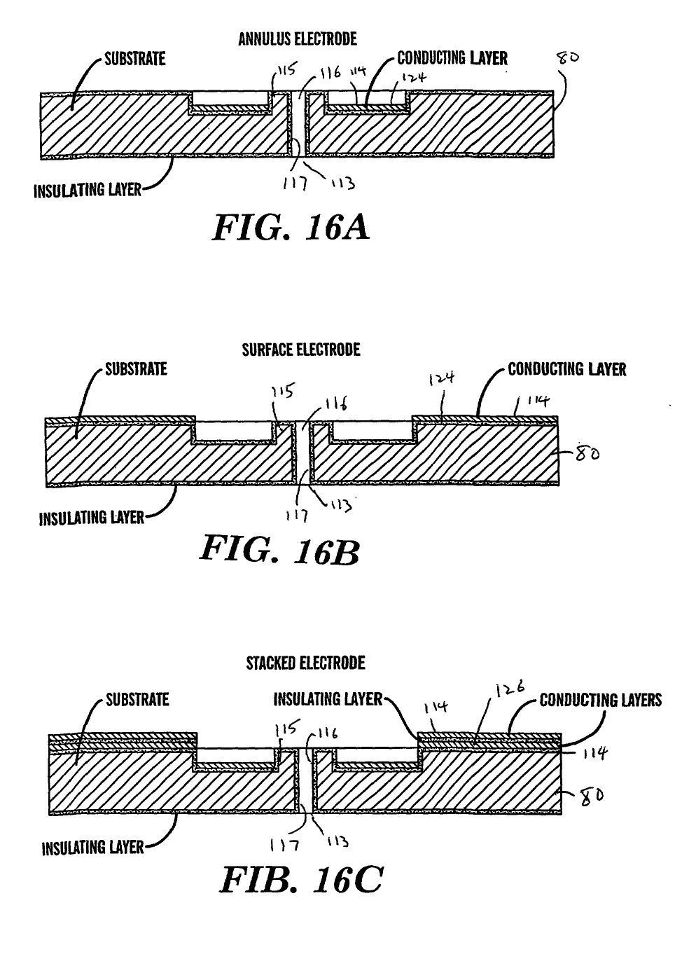

Other voltages may also be provided by the chip holder 125, as shown in FIG. 15. Additional examples for the application of substrate voltage required, control voltages on electrodes on the front surface or in layers 126 in the chip either for the whole chip, or around each electrospray device, or groups of devices is illustrated in FIGS. 16A-C. These voltages may be used to steer ions, dispel space charge, and dispel surface charge, thus maximizing sensitivity of the electrospray device.

The fluid probe may include a chromatographic column, desalting column, or other stationary phase, including a packed material or surface coating. The fluid probe may also be a capillary tube sample container or larger internal diameter sample container. The fluid probe may also be an electrically conductive pipette tip, such as a pipette tip made from graphite impregnated polypropylene. The fluid probe may be reusable or disposable itself or have a reusable or disposable tip.

Electrospray occurs because of the generation of a controlled electric field between the fluid and the substrate of the chip. The chip holder can supply voltage to the substrate of the chip. When the chip holder is electrically conductive the holder may be tied to ground potential and the substrate voltage is simply applied by holding the edge of chip to the chip mount. This can be done by any known method, for example, mechanically or by using a conductive paste or epoxy. More particularly, the chip holder can supply electrospray voltage to the fluid at the chip, either to individual nozzles or all nozzles at once. Alternately, the delivery probe/column/sample capillary can be used to provide the electrospray voltage. A small probe that is attached to, but electrically insulated from, and moves with the fluid probe may be used to provide the electrospray voltage, either individually or all together or in groups. This also provides some degree of isolation of column/probe from the electrospray voltage, so less electro-osmosis or electro-chromatography is provided.

Individual conducting pads can be applied on the back of the chip to individually apply voltage to each nozzle. Similarly, metal coatings can be applied on the front of the chip to apply voltage to each nozzle.

Since the electric field around each nozzle is preferably defined by the fluid and substrate voltage at the nozzle tip, multiple nozzles can be located in close proximity, on the order of tens of microns. This allows for the formation of multiple electrospray plumes from multiple nozzles of a single fluid stream thus greatly increasing the electrospray sensitivity available for microchip-based electrospray devices. Multiple nozzles of an electrospray device in fluid communication with one another not only improve sensitivity but also increase the flow rate capabilities of the device. For example, the flow rate of a single fluid stream through one nozzle having the dimensions of a 10 micron inner diameter, 20 micron outer diameter, and a 50 micron length is about 1 μL/min.; and the flow rate through 200 of such nozzles is about 200 μL/min. Accordingly, devices can be fabricated having the capacity for flow rates up to about 2 μL/min., from about 2 μL/min. to about 1 mL/min., from about 100 nL/min. to about 500 nL/min., and greater than about 2 μL/min. possible.

Arrays of multiple electrospray devices having any nozzle number and format may be fabricated. The electrospray devices can be positioned to form from a low-density array to a high-density array of devices. For example, arrays can be provided having a spacing between adjacent devices of 9 mm, 4.5 mm, 2.25 mm, 1.12 mm, 0.56 mm, 0.28 mm, and smaller to a spacing as close as about 50 μm apart, respectively, which correspond to spacing used in commercial instrumentation for liquid handling or accepting samples from electrospray systems. Similarly, systems of electrospray devices can be fabricated in an array having a device density exceeding about 5 devices/cm², exceeding about 16 devices/cm², exceeding about 30 devices/cm², and exceeding about 81 devices/cm², preferably from about 30 devices/cm² to about 100 devices/cm².

Dimensions of the electrospray device can be determined according to various factors such as the specific application, the layout design as well as the upstream and/or downstream device to which the electrospray device is interfaced or integrated. Further, the dimensions of the channel and nozzle may be optimized for the desired flow rate of the fluid sample. The use of reactive-ion etching techniques allows for the reproducible and cost effective production of small diameter nozzles, for example, a 2 μm inner diameter and 5 μm outer diameter. Such nozzles can be fabricated as close as 20 μm apart, providing a density of up to about 160,000 nozzles/cm². Nozzle densities up to about 10,000/cm², up to about 15,625/cm², up to about 27,566/cm², and up to about 40,000/cm², respectively, can be provided within an electrospray device. Similarly, nozzles can be provided wherein the spacing on the ejection surface between the centers of adjacent exit orifices of the spray units is less than about 500 μm, less than about 200 μm, less than about 100 μm, and less than about 50 μm, respectively. For example, an electrospray device having one nozzle with an outer diameter of 20 μm would respectively have a surrounding sample well 30 μm wide. A densely packed array of such nozzles could be spaced as close as 25 μm apart as measured from the nozzle center.

For example, in one currently preferred embodiment the silicon substrate of the electrospray device is approximately 250-500 μm in thickness and the cross-sectional area of the through-substrate channel is less than approximately 2,500 μm². Where the channel has a circular cross-sectional shape, the channel and the nozzle have an inner diameter of up to 50 μm, more preferably up to 30 μm; the nozzle has an outer diameter of up to 60 μm, more preferably up to 40 μm; and nozzle has a height of (and the annular region has a depth of) up to 100 μm. The recessed portion preferably extends up to 300 μm outwardly from the nozzle. The silicon dioxide layer has a thickness of approximately 1-4 μm, preferably 1-3 μm. The silicon nitride layer has a thickness of approximately less than 2 μm. The autosampler of the present invention can be fabricated to interface with electrospray devices having the above-noted nozzle density and flow rates so as to automate the sampling process and achieve the benefits of such high-density systems.

Furthermore, the electrospray device may be operated to produce larger, minimally-charged droplets. This is accomplished by decreasing the electric field at the nozzle exit to a value less than that required to generate an electrospray of a given fluid. Adjusting the ratio of the potential voltage of the fluid and the potential voltage of the substrate controls the electric field. A fluid to substrate potential voltage ratio approximately less than 2 is preferred for droplet formation. The droplet diameter in this mode of operation is controlled by the fluid surface tension, applied voltages and distance to a droplet receiving well or plate. This mode of operation is ideally suited for conveyance and/or apportionment of a multiplicity of discrete amounts of fluids, and may find use in such devices as ink jet printers and equipment and instruments requiring controlled distribution of fluids.

Although the invention has been described in detail for the purpose of illustration, it is understood that such detail is solely for that purpose, and variations can be made therein by those skilled in the art without departing from the spirit and scope of the invention which is defined by the following claims.

What is claimed is:

1. A robot autosampler, comprising:
a probe carriage being movable between a sample source and an electrospray chip holder and comprising at least one fluid delivery probe which accepts sample from the source and discharges sample to a chip in the chip holder;
an electrospray chip holder; and
an alignment system which aligns the probe with the chip holder and the chip holder with a detector.

2. The robot autosampler of claim 1, further comprising a voltage probe electrically insulated from and mounted to said fluid delivery probe.

3. The robot autosampler of claim 1, further comprising an electrospray chip mounted to said chip holder.

4. The robot autosampler of claim 3, further comprising a detector in electrospray communication with said electrospray chip.

5. The robot autosampler of claim 4, wherein said detector comprises a mass spectrometer.

6. The robot autosampler of claim 1, wherein said fluid delivery probe comprises a chromatographic column or desalting column.

7. The robot autosampler of claim 1, wherein said fluid delivery probe comprises a capillary tube sample container or larger internal diameter sample container.

8. The robot autosampler of claim 1, wherein said fluid delivery probe comprises a reusable probe, disposable probe, reusable tip, or disposable tip.

9. The robot autosampler of claim 1, wherein said chip holder provides electrospray voltage to the substrate of the chip through the chip mount.

10. The robot autosampler of claim 1, wherein said chip holder provides voltage or ground potential to the substrate of the chip, to at least one nozzle to provide or control electrospray.

11. The robot autosampler of claim 1, wherein said fluid delivery probe provides electrospray voltage to the fluid.

12. The robot autosampler of claim 2, wherein said voltage probe provides electrospray voltage to the surface of the chip, independently to individual nozzles, groups of nozzles, or all nozzles at once.

13. The robot autosampler of claim 3, wherein said electrospray chip further comprises a plurality of individual conducting pads applied on the back of the chip to apply voltage.

14. The robot autosampler of claim 3, wherein said electrospray chip further comprises metal coatings applied on the front of the chip to apply voltage.

15. The robot autosampler of claim 1, wherein said fluid delivery probe further comprises a seal which prevents leakage during delivery of the fluid to the chip.

16. The robot autosampler of claim 3, wherein said electrospray chip comprises a plurality of electrospray devices, each generating one or a multiple of electrospray plumes when activated.

17. The robot autosampler of claim 3, wherein said electrospray chip comprises multiple electrospray devices grouped in a high-density array, each generating one or a multiple of electrospray plumes when activated.

18. The robot autosampler of claim 1, further comprising an assembler control unit in communication with the autosampler.

19. A method for automated manipulation of multiple samples for generation of multiple electrosprays in communication with a detector, comprising:
providing a robot autosampler, which can be programmed to engage a tip onto a fluid delivery probe, load the tip with sample containing at least one electrolyte, transfer the sample loaded tip to communicate with an electrospray chip containing at least one electrospray device, electrospray the at least one analyte, discard the used tip, and engage another tip onto the probe to repeat the loading, transferring, and electrospraying cycle;
engaging a tip onto the autosampler probe;
loading the probe tip with a sample containing at least one analyte;
transferring the at least one analyte to at least one electrospray device on the electrospray chip;
electrospraying the at least one analyte from at least one electrospray device on the electrospray chip;
manipulating the electrospray chip in communication with a detector in a manner to detect analyte from the electrospray, and
repeating the engaging, loading, transferring, and electrospraying cycle.

20. The method of claim 19, wherein said detector is a mass spectrometer.

21. The method of claim 19, wherein said tip is pre-loaded with a sample containing at least one analyte.

22. The method of claim 19, wherein said tip is reused.

23. The method of claim 19, wherein control voltages are applied to the electrospray device by the autosampler.

24. The method of claim 19, wherein said automated manipulation is controlled by programmable computer software.
